(19) United States
(12) Patent Application Publication
(10) Pub. No.: US 2016/0106480 A1
(43) Pub. Date: Apr. 21, 2016

(54) EXTENSION PIECE AT SCREW END OF PEDICLE SCREW, DILATOR AND PUSH ROD RESET LEVER

(71) Applicants: Yue ZHOU, Shanghai (CN); Michael Mingyan LIU, Shanghai (CN); Wenqiao ZHANG, Shanghai (CN)

(72) Inventors: Yue ZHOU, Shanghai (CN); Michael Mingyan LIU, Shanghai (CN); Wenqiao ZHANG, Shanghai (CN)

(21) Appl. No.: 14/440,252

(22) PCT Filed: Jul. 2, 2014
(86) PCT No.: PCT/CN2014/081528

§ 371 Date: May 1, 2015

(51) Int. Cl. A61B 17/70

(52) U.S. Cl. CPC A61B 17/7085; A61B 17/7032; A61B 17/7002; A61B 17/7086

(57) ABSTRACT

Disclosed is an extension piece at a screw end of a pedicle screw, where two extension pieces are oppositely disposed at the pedicle screw tail, and the extension pieces are flexible extension pieces. The extension pieces are flexibly designed, having elastically bendable characteristics, the extension pieces in the subcutaneous part can be elastically bent by external force, its dilation trend may not extend to the epithelial part, and it is feasible that muscles are squeezed only subcutaneously, so as to keep small incisions, and in the case of a threading operation of a reset rod, as portions of the two extension pieces in the subcutaneous part are dilated and separated after bending, a sufficient threading space can be provided, so that the threading operation is easy to perform, thereby reducing risks of surgeries. Further disclosed are a dilator and a push rod reset lever cooperating with the extension piece.

### BACKGROUND OF THE INVENTION

1. Field of the Invention

The present invention relates to the field of medical instrument technologies, and more particularly to an extension piece at a screw end of a pedicle screw, and a dilator and a push rod reset lever cooperating with the extension piece.

2. Related Art

A technology of internal fixation of spine can be simply summarized into two steps: first whole, then local. That is, a reset rod is first pre-bent into a bending shape of human spine and is connected with the spine through a pedicle screw or the like; the reset rod is further stretched/pressurized, and the reset rod is finally locked to the pedicle screw.

Steps of surgical operations of the technology of internal fixation of spine are as follows:

1. preoperative preparation: select (length and diameter of) a pedicle screw;

2. positioning the pedicle screw and preparing a screw path;

3. implanting the pedicle screw into a human body;

4. placing a rod; generally, the tail of the pedicle screw has a U-shaped groove whose opening is outward, and “placing a rod” means placing a reset rod into the U-shaped groove of the pedicle screw;

5. pressing the reset rod in the U-shaped groove, and performing temporary locking. The step is mainly for pressing the reset rod to the bottom of the U-shaped groove of the pedicle screw, and pre-locking the reset rod at the same time; a tool that presses the rod includes push rods, frog clamps and the like;

6. stretching or pressurizing spine along a direction of the reset rod; and

7. making final locking; the reset rod is locked in the U-shaped groove through a screw plug, a groove wall of the U-shaped groove has threads, and the screw plug fits with the threads to lock the reset rod.

The whole process of an open surgery is described as above. In the open surgery, placing, pressing and other operations are directly performed at the opening, therefore, a large incision is required, but the large incision may cause permanent damage to some soft tissues of the human body, and postoperative recovery time is longer.

In view of the disadvantages of the open surgery, a minimally invasive therapy emerges, which mainly relies on extension pieces at the tail of a pedicle screw. The tail of the pedicle screw has a U-shaped groove, two opposite sidewalls of the U-shaped groove each have an extending rigid extension piece, that is, the two extension pieces are disposed oppositely, in the case of a small incision, one part of the extension pieces is located outside the skin, and the other part thereof is located inside the skin. The placing and pressing operations can be performed under the guide of the extension pieces without large incisions. When the placing operation is implemented by means of the extension pieces, the reset rod is threaded from between one extension piece of the pedicle screw to between the other extension piece.

However, the threading operation in the minimally invasive therapy is difficult. The extension pieces cannot be bent and deformed during the surgery, and a space between the two extension pieces for threading of the reset rod is unchangeable, resulting in that the threading operation of the reset rod is very difficult and risky.

In addition, during the pressing operation, the push rod needs to be provided with two guide bars, which are rigidly connected with the extension pieces, axial movement of a pressure lever of the push rod requires the guide bars, to ensure a movement direction, so as to press the rest rod; the push rod of the structure is complicated, is not easy to operate, and has a greater surgery risk.

In view of this, how to improve the extension piece at the tail of the pedicle screw, so that the threading operation can be performed smoothly in the minimally invasive therapy, to reduce the surgery risk is a technical problem to be solved by persons skilled in the art.

### SUMMARY OF THE INVENTION

To solve the above technical problem, the object of the present invention is to provide an extension piece at a screw end of a pedicle screw, a dilator and a push rod reset lever. The extension piece can provide a sufficient threading space on the premise of ensuring a minimally invasive incision, so as to reduce the risk of the minimally invasive surgery.

According to the extension piece at a screw end of a pedicle screw, two extension pieces are oppositely disposed at the tail of the pedicle screw, and the extension pieces are flexible extension pieces.

Preferably, one end of the extension pieces is provided with a snap hole for a snap-in of a transverse boss at the screw end of the pedicle screw; the end of the extension pieces is further provided with a flexible snap strip opposite to the snap hole, and the snap strip forms a snap ring for a snap-in of an axial boss at the screw end of the pedicle screw with two sides of the extension pieces.

Preferably, two sides of one end of the extension pieces are both provided with a bending sidewall, the end refers to the end of the extension pieces provided with the snap hole; the snap strip forms the snap ring with the two bending sidewalls; and contact surfaces of the snap strip and the axial boss at the screw end of the pedicle screw are matched with each other.

Preferably, an end portion on the extension pieces away from the pedicle screw is provided with a boss extending outwardly.

According to the extension piece in the embodiment, in the case of a minimally invasive surgery with a small incision, first implant a pedicle screw installed with extension pieces into spine from the small incision; then, dilate and separate the part of the extension pieces below the skin, as the extension pieces are flexible, its dilation trend may not extend to the epithelial part, and it is feasible that muscles are squeezed only subcutaneously, so as to keep small incisions; then perform a threading operation of the reset rod, as the two extension pieces in the subcutaneous part are dilated and separated, a sufficient threading space is provided, so that the threading operation is easy to perform, thereby reducing the risk of the minimally invasive surgery.

The present invention further provides a dilator for dilating the extension piece, where the dilator includes two hinged rods; each of the rod includes a dilation head and a dilation leg connected with each other, and their connection position is a hinge position; the two dilation legs can extend between the two extension pieces, and the two dilation heads can open and close by external force to drive ends of the dilation legs to be near or away; and portions of the two extension pieces inside the skin can be dilated and separated when the ends of the dilation legs are away.

The dilator can manipulate the dilation legs to dilate the part of the extension pieces below the skin outside the skin, to achieve the object of expanding the threading space without affecting the small incision, thereby reducing the risk of the minimally invasive surgery.

Preferably, a spring is disposed between the two dilation heads.

Preferably, the two dilation heads are both connected with a dilation handle.

Preferably, the dilator further includes a housing covering the dilation heads, the spring and the hinge position, two sides of the housing are provided with a snap cover for insertion of the extension pieces.

Preferably, the ends of the dilation legs each have a clamp for insertion of the extension pieces, and the clamp has an opening.

Preferably, the two dilation legs are crossed.

The present invention further provides a push rod reset lever, cooperating with the extension piece to press a reset rod, where the push rod reset lever includes a pressure lever and a handheld portion that can restrict the two extension pieces to move along a push rod direction, the pressure lever can be inserted into the handheld portion and slides axially relative to the handheld portion; and two sides of one end of the pressure lever are both provided with a cannula, respectively for insertion of the two extension pieces and screw groove sidewalls of the pedicle screw.

The push rod reset lever cooperates with the flexible extension pieces, axially slides along the extension pieces, and finally contacts the reset rod, and in the case of continuous application of force, the push rod reset lever can sleeve a sidewall of a screw groove of the pedicle screw, so as to press the reset rod to the bottom of the screw groove. The push rod reset lever cooperates with the flexible extension pieces, uses the extension pieces as guide bars, to simply implement the pressing operation, and can restore the extension pieces to a pre-dilation state during sliding, and when the rod is pressed to the bottom of the screw groove, the cannula of the pressure lever of the push rod reset lever sleeves the sidewall of the screw groove externally, to implement the subsequent resetting operation (stretch/pressurization).

Preferably, an outer wall of the pressure lever is provided with pawls, and the handheld portion is provided with latches that can be snapped into gaps between adjacent pawls.

Preferably, two sides of the handheld portion are both provided with a side hole, an end portion on the extension pieces away from the pedicle screw is provided with a boss extending outwardly, and the side hole of the handheld portion is for a snap-in of the boss.

Preferably, one end of the pressure lever provided with the cannula has a concave arc surface that fits with a surface of the reset rod.

### BRIEF DESCRIPTION OF THE DRAWINGS

FIG. 1 is a schematic structural view of a specific embodiment of an extension piece at a screw end of a pedicle screw according to the present invention;

FIG. 2 is a schematic structural view from another angle of the extension piece in FIG. 1;

FIG. 3 is a schematic partially enlarged view of Site A in FIG. 2;

FIG. 4 is a schematic state view of a process of installing the extension piece in FIG. 1 to the tail of the pedicle screw;

FIG. 5 is a schematic view after the extension piece in FIG. 1 is installed to the screw end of the pedicle screw;

FIG. 6 is a schematic partially enlarged view of Site B in FIG. 4;

FIG. 7 is a schematic partially enlarged view of Site C in FIG. 5;

FIG. 8 is a schematic structural view of a specific embodiment of a dilator according to the present invention;

FIG. 9 is a schematic structural view after a housing is installed to the dilator in FIG. 8;

FIG. 10 is a schematic view showing that the dilator in FIG. 8 cooperates with the extension piece and the extension piece has not stretched yet;

FIG. 11 is a schematic structural view after the dilator in FIG. 8 dilates to stretch the extension piece;

FIG. 12 is a schematic structural view of a specific embodiment of a push rod reset lever according to the present invention;

FIG. 13 is a schematic view of a pressing process of the push rod reset lever in FIG. 12;

FIG. 14 is a schematic structural view after completion of pressing of the push rod reset lever in FIG. 12;

FIG. 15 is a schematic partially enlarged view of Site D in FIG. 13;

FIG. 16 is an axial sectional view of the push rod reset lever in FIG. 12; and

FIG. 17 is a schematic partially enlarged view of Site E in FIG. 16.

In the figures,

1 extension piece, 11 snap hole, 12 snap strip, 13 boss, 14 bending sidewall, 2 pedicle screw, 21 U-shaped groove, 211 transverse boss, 212 axial boss, 3 dilator, 31 rod, 311 dilation head, 312 dilation leg, 313 hinge position, 32 dilation handle, 33 spring, 312a clamp, 34 housing, 341 snap cover, 4 push rod reset lever, 41 pressure lever, 411 pawl, 412 cannula, 42 handheld portion, 421 latch, 422 side hole, 5 reset rod

### DETAILED DESCRIPTION OF THE EMBODIMENTS

To make persons skilled in the art better understand the technical solution of the present invention, the present invention is further described below in detail with reference to the accompanying drawings and specific embodiments.

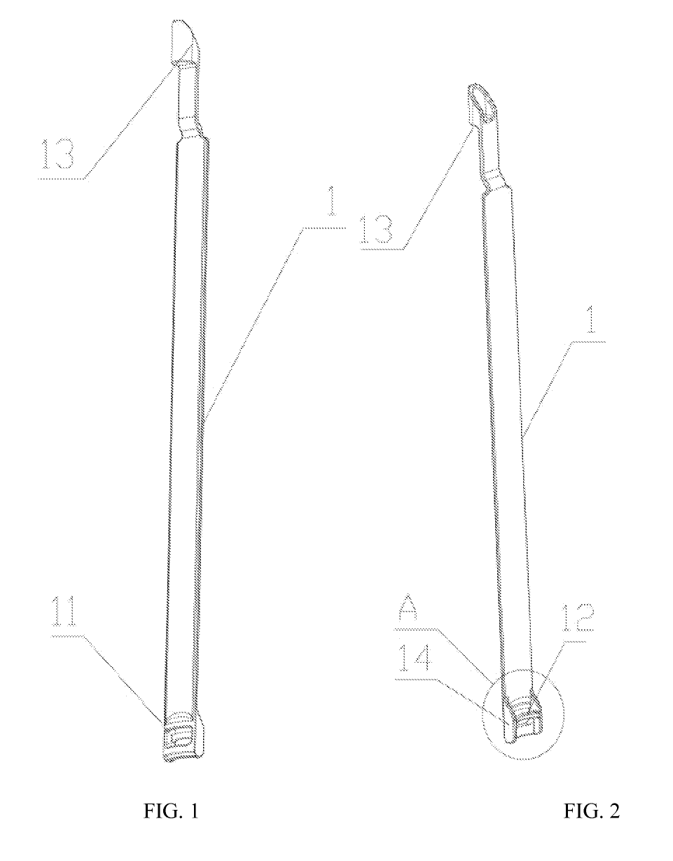

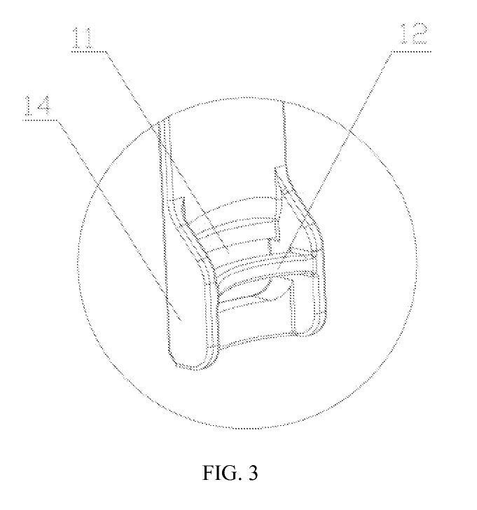

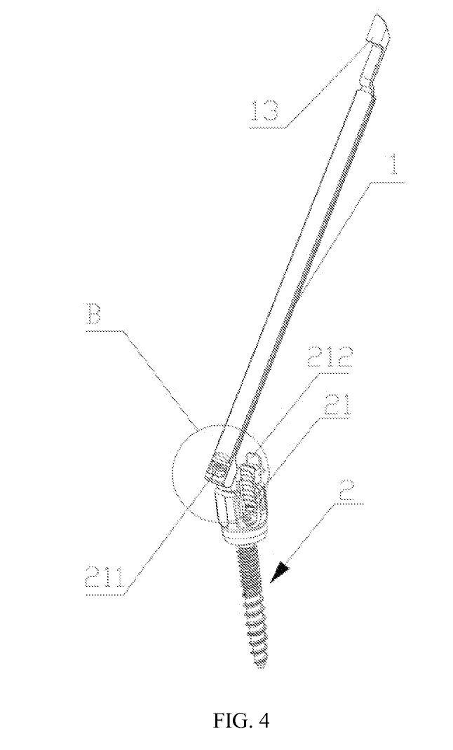

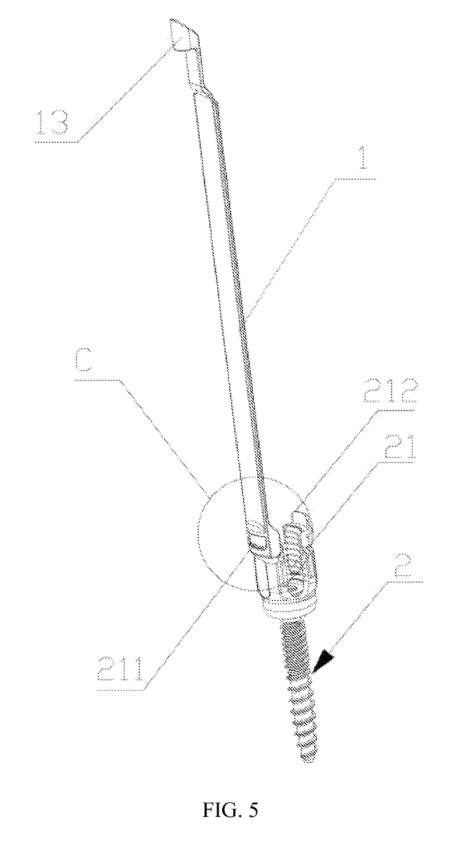

Referring to FIGS. 1-5, FIG. 1 is a schematic structural view of a specific embodiment of an extension piece at a screw end of a pedicle screw according to the present invention; FIG. 2 is a schematic structural view from another angle of the extension piece in FIG. 1; FIG. 3 is a schematic partially enlarged view of Site A in FIG. 2; FIG. 4 is a schematic state view of a process of installing the extension piece in FIG. 1 to the tail of the pedicle screw; and FIG. 5 is a schematic view after the extension piece in FIG. 1 is installed to the screw end of the pedicle screw.

The screw end of the pedicle screw 2 generally has a screw groove, such as a U-shaped groove 21 as shown, to place a reset rod 5; two extension pieces 1 are installed to two sidewalls of the screw groove respectively, that is, sidewalls on two sides of the U-shaped groove 21 as shown, the two extension pieces 1 are oppositely disposed at the tail of the pedicle screw 2, FIGS. 4 and 5 only show the extension piece 1 on one side, and the extension piece 1 is a flexible extension piece, that is, the extension piece 1 has elastically bendable characteristics, and is deformable by external force.

The extension piece 1 in this embodiment is of an elongated structure, and it can be seen from FIGS. 4-5 that, the length of the extension piece 1 is much greater than that of the pedicle screw 2. The manner in which the extension piece 1 of the structure connects with the pedicle screw 2 can specifically be an insertion manner.

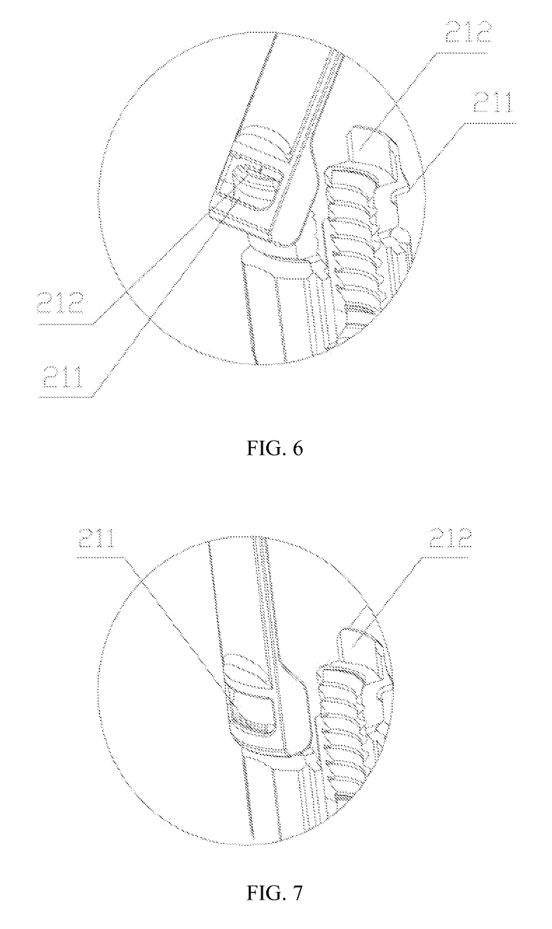

As shown in FIGS. 6-7, FIG. 6 is a schematic partially enlarged view of Site B in FIG. 4; and FIG. 7 is a schematic partially enlarged view of Site C in FIG. 5.

Sidewalls of the U-shaped groove 21 of the pedicle screw 2 have an axial boss 212 and a transverse boss 211, the body on one end of the extension piece 1 is provided with a snap hole 11 for snap-in of the transverse boss 21 lat the screw end of the pedicle screw 2; in addition, the end of the extension piece 1 is further provided with a flexible snap strip opposite to the snap hole 11, and the snap strip 12 forms a snap ring for snap-in of the axial boss 212 at the screw end of the pedicle screw 2 with two sides of the extension pieces 1. As shown in FIG. 4, at first, the extension piece 1 is tilted to a certain angle (tilted towards the other extension piece 1), the axial boss 212 can be inserted into the snap hole 11 of the extension piece 1, then, the extension piece 1 is turned towards an outer side (the outer side herein refers to a direction away from an axis of the pedicle screw 2) to make the extension piece 1 parallel to an axial direction of the pedicle screw 2, and during turning, due to the elastically bendable characteristics of the extension piece 1, certain elastic deformation may be produced during turning, the transverse boss 211 is finally snapped in the snap hole 11, and the axial boss 212 is finally inserted into the snap ring, that is, the assembling process in FIGS. 4-5.

Further, two sides of one end of the extension pieces 1 provided with the snap hole 11 can both be provided with a bending sidewall 14, the flexible snap strip 12 forms a snap ring with the two bending sidewalls 14, as shown in FIG. 3, and in this way, the extension piece 1 can form the snap ring integrally, which is easy to machine. In addition, contact surfaces of the snap strip 12 and the axial boss 212 at the screw end of the pedicle screw 2 are matched with each other. It can be seen from FIG. 3 that, a surface of the snap strip 12 for contacting the axial boss 212(a surface opposite to the snap hole 11) is an arc surface, which is matched with an arc outer wall of the axial boss 212.

The extension piece 1 and the pedicle screw 2 after being installed in place in the above insertion manner cannot be normally disassembled, and can only be destructively disassembled through specialized tools, therefore, the extension piece 1 can be fixed to the pedicle screw 2 all the time during surgery, so as to ensure security of the surgery. The extension piece 1 can also be connected with the screw end of the pedicle screw 2 in other connecting manners, for example, clamping, welding, threaded connection and the like; certainly, the above insertion manner is a more optimal solution.

When the extension piece 1 in this embodiment is during surgery, part of the extension piece 1 can be outside the skin, part of the extension piece 1 and the pedicle screw 2 are inside the skin together, as the extension piece 1 is a flexible extension piece, portions of the two extension pieces 1 inside the skin can be bent and deformed by external force, and the two extension pieces 1 can be deformed along opposite directions, so that the portions of the two extension pieces 1 inside the skin can be dilated and separated.

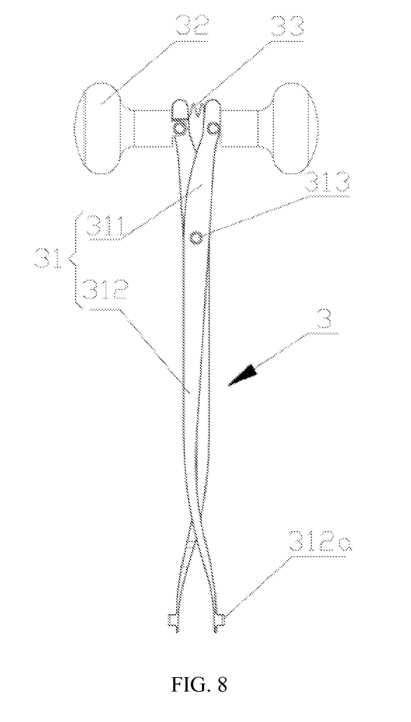

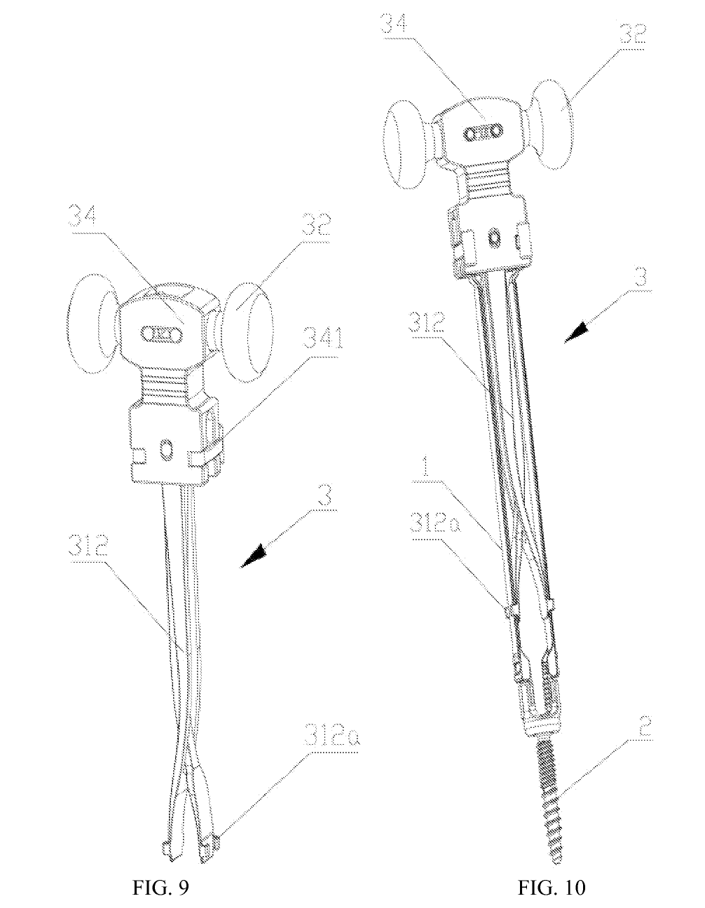

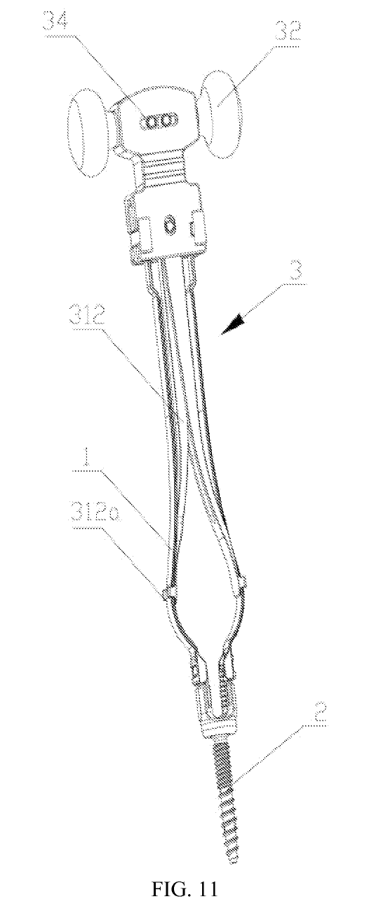

Referring to FIGS. 8-11, FIG. 8 is a schematic structural view of a specific embodiment of a dilator according to the present invention; FIG. 9 is a schematic structural view after a housing is installed to the dilator in FIG. 8; FIG. 10 is a schematic view showing that the dilator in FIG. 8 cooperates with the extension piece and the extension piece has not stretched yet; and FIG. 11 is a schematic structural view after the dilator in FIG. 8 dilates to stretch the extension piece.

The dilator 3 includes two hinged rods 31, similar to a clamp structure. Each of the rods 31 includes a dilation head 311 and a dilation leg 312 connected with each other, their connection position is a hinge position 313, and as shown in FIG. 8, the two dilation heads 311 can open and close by external force to drive ends of the two dilation legs 312 to be near or away. During surgery, the two dilation legs 312 are extended between the two extension pieces 1, as shown in FIGS. 10 and 11, and the ends of the dilation legs 312 are extended below the skin, the two dilation heads 311 are then gripped to make them close to each other, the ends of the two dilation legs 312 are away from each other, and as the extension pieces 1 are flexibly designed, the ends of the dilation legs 312 intended to be away can drive portions of the two extension pieces 1 inside the skin to be dilated and separated. As shown in FIG. 11, lower ends of the two extension pieces 1 are stretched by the two dilation legs 312 to be elastically bent.

According to the extension piece 1 in this embodiment, in the case of a minimally invasive surgery with a small incision:

firstly, implant the pedicle screw 2 installed with the extension pieces 1 into spine from the small incision;

then, dilate and separate the part of the extension pieces 1 below the skin through the dilator 3, as the extension pieces 1 are flexibly designed, its dilation trend may not extend to the epithelial part, and it is feasible that muscles are squeezed only subcutaneously, so as to keep small incisions; and

next, perform a threading operation of the reset rod 5, as the two extension pieces 1 in the subcutaneous part are dilated and separated, a sufficient threading space is provided, so that the threading operation is easy to perform, thereby reducing the risk of the minimally invasive surgery.

With respect to the dilator 3 in this embodiment, further improvement may be made.

As shown in FIG. 8, each of the two dilation heads 311 is connected with a dilation handle 32. The dilation heads 311 as a part of the rods 31 are not easy to grip during manual operation, and operation is easy when the dilation handle 32 is set. The two dilation handles 32 in FIG. 8 are located at two sides of the two dilation heads 311 respectively, and is drum-shaped, so as to improve handheld comfort, and easily manipulate the opening and closing of the dilation legs 312.

A spring 33 may further be disposed between the two dilation heads 311, as shown in FIG. 8. After the spring 33 is set, without external force, gaps of the dilation legs 312 is stable, and in the process that the dilator 3 cooperates with the extension pieces 1, the extension pieces 1 may not be accidentally dilated due to an unstable state of the dilation legs 312 to affect the incision; in addition, due to existence of the spring 33, when force is applied to the dilation handles 32, they will be subject to resistance from the spring 33, which helps medical staff to control the degree of opening of the dilation legs 312, to prevent errors caused by excessive force.

In addition, the dilator 3 may further be provided with a housing 34 covering the dilation heads 311, the spring 33 and the hinge position 313, and the housing 34 plays a role of protection and support, and can also enhance strength of the dilator 3, and prolong the service life, as shown in FIG. 9. Besides, two sides of the housing 34 may be further provided with a snap cover 341 for insertion of the extension pieces 1. When the extension pieces 1 cooperate with the dilator 3, it is necessary to ensure that the dilation legs 312 on the dilator 3 can stretch the extension pieces 1 when being separated; to this end, the dilator 3 and the extension pieces 1 preferably have a positioning relationship. After an end portion on the extension pieces 1 away from the screw end is snapped into the snap cover 341 of the dilator 3, a position relationship between the extension pieces 1 and the dilator 3 is determined, which ensures that the extension pieces 1 can follow but are not separated from the dilation legs 312 to some extent.

Further, in order to strengthen the positioning relationship between the dilator 3 and the extension pieces 1, ends of the dilation legs 312 each can be provided with a clamp 312a for insertion of the extension pieces 1, where the clamp 312a has an opening, as shown in FIG. 9, and the opening 312a facilitates insertion of the extension pieces 1. The ends of the dilation legs 312 are located below the skin, observation is difficult, and the clamp 312a ensures that the dilation legs 312 can stretch the extension pieces 1. A preferred solution is that the clamp 312a and the snap cover 341 are jointly disposed, the clamp 312a can fix the lower ends of the extension pieces 1, the snap cover 341 can fix upper ends of the extension pieces 1, and when both the snap cover 341 and the clamp 312a are disposed, more reliable and stable cooperation between the extension pieces 1 and the dilator 3 is achieved.

In addition, it can be seen from FIGS. 8 and 9 that the two dilation legs 312 of the dilator 3 are crossed. As opening and closing of the dilation heads 311 of the dilator 3 can drive the dilation legs 312 to open and close, in fact, upper ends and ends of the two rods 31 are crossed. After the two dilation legs 312 are crossed, when the two dilation heads 311 are close, portions of the two dilation legs 312 above the crossing point are gradually close, and portions of the ends below the crossing point are gradually away. Thus, after the two dilation legs 312 are crossed, when ends of the dilation legs 312 are dilated to stretch the extension pieces 1, width of other portions of the dilation legs 312 gradually decreases, as shown in FIG. 11, and in this way, dilation of the ends of the dilation legs 312 may not cause dilation of the other portions of the dilation legs 312, so as to further ensure that the small incision is not affected.

It can be imaged that, when the two dilation legs 312 of the dilator 3 are not crossed, the hinge position between the dilation legs 312 and the dilation heads 311 should be substantially at the incision, and in this way, the dilation legs 312 affect the incision less during dilation, so as to meet the requirements of the small incision of the minimally invasive surgery.

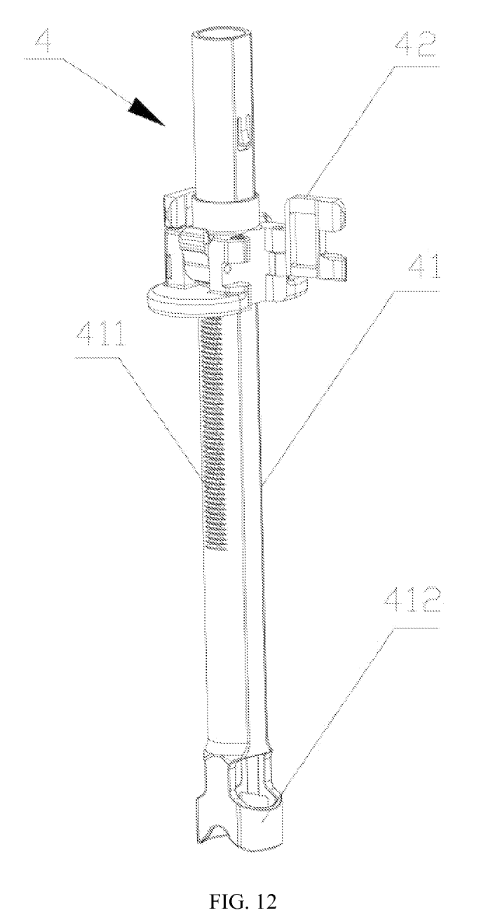

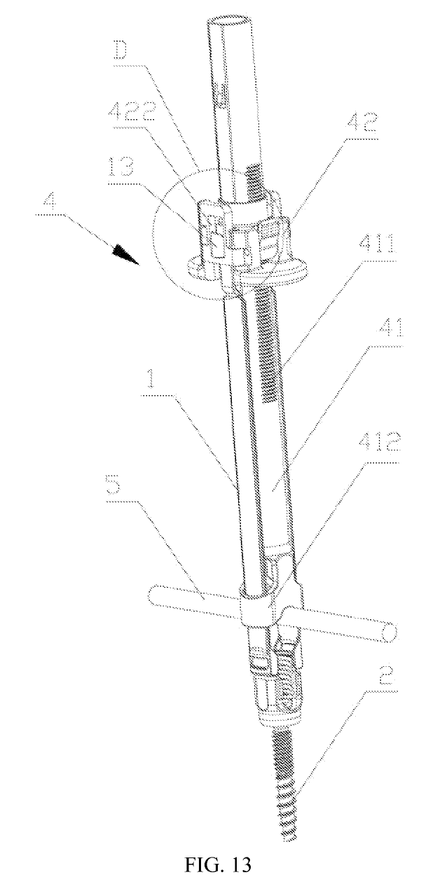

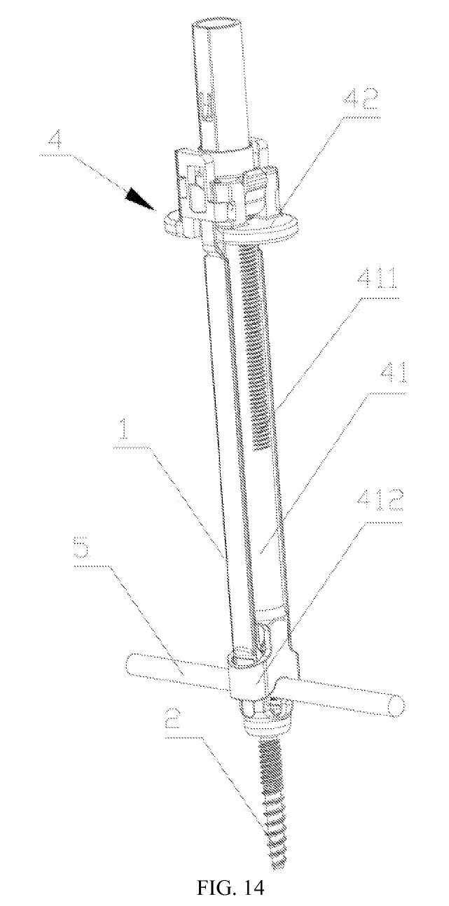

After the threading operation ends, it is necessary to press the rod, this embodiment uses a specialized push rod reset lever 4 to cooperate with the extension piece 1 in the above embodiment for the pressing operation; please referring to FIGS. 12-14, FIG. 12 is a schematic structural view of a specific embodiment of a push rod reset lever according to the present invention; FIG. 13 is a schematic view of a pressing process of the push rod reset lever in FIG. 12; and FIG. 14 is a schematic structural view after completion of pressing of the push rod reset lever in FIG. 12.

The push rod reset lever 4 includes a pressure lever 41 and a handheld portion 42 that can restrict the two extension pieces 1 to move along a push rod direction. The pressure lever 41 can be inserted into the handheld portion 42 and slides axially relative to the handheld portion 42, the handheld portion 42 has an axial through hole, and the pressure lever 41 can be inserted into the axial through hole. Two sides of one end of the pressure lever 41 are provided with a cannula 412, respectively for insertion of the two extension pieces 1, and the cannulas 412 and the pressure lever 41 can be integrally machined.

During the pressing operation, the two cannulas 412 at one end of the pressure lever 41 first sleeve the two extension pieces 1 respectively, the pressure lever 41 is located between the two extension pieces 1, and one end on the two extension pieces 1 away from the pedicle screw 2 is connected with the handheld portion 42; the handheld portion 42 is gripped, the pressure lever 41 is axially moved relative to the handheld portion 42, to be continuously pressed to the reset rod 5, force is continuously applied until the reset rod 5 is pressed to the bottom of the U-shaped groove 21 of the pedicle screw 2, at this time, the cannula 412 of the end portion of the pressure lever 41 directly sleeves the sidewall of the U-shaped groove 21, and the cannula 412 and the sidewall of the U-shaped groove 21 can be fixed, for example, in a manner of rotating snap and the like, to achieve rigid constraint, as shown in FIG. 14, so as to implement the subsequent resetting operation (stretch/pressurization).

Herein, the function of gripping the handheld portion 42 lies in that, without affecting the pedicle screw 2 and spine, the handheld portion 42 is gripped to maintain the state of the extension pieces 1 and the pedicle screw 2 unchanged, so as to provide support for the push rod, and otherwise, force of the push rod may act upon the pedicle screw 2, and then acts upon the spine, which may damage the spine and cannot achieve the pressing effect. So, the push rod reset lever 4 cooperates with the flexible extension pieces 1, so as to smoothly achieve the pressing operation of the reset rod 5.

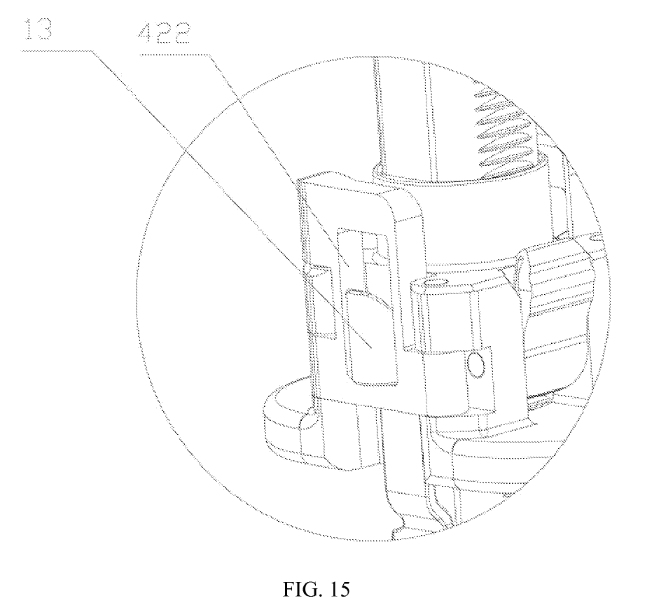

In order to achieve that the extension pieces 1 are in a stable state when the handheld portion 42 is gripped, one end on the extension pieces 1 away from the pedicle screw 2 can be provided with a boss 13 extending outwardly, as shown in FIG. 1 and FIG. 15, wherein FIG. 15 is a schematic partially enlarged view of Site D in FIG. 13. The handheld portion 42 has a side hole 422 correspondingly for snap-in of the boss 13, as shown in FIGS. 12-14. When the push rod reset lever 4 is installed and cooperates with the extension pieces 1 as a whole, the boss 13 of the extension pieces 1 can be snapped into the side hole 422 of the handheld portion 42, and when the handheld portion is gripped, the boss 13 interferes with walls of the side hole 422, which restricts the extension pieces 1 to move along a push rod direction, thereby providing support for the push rod.

The push rod reset lever 4 cooperates with the flexible extension pieces 1 to make axial movement, it can be seen from figures that the extension pieces 1 are of an elongated structure, that is, the extension pieces 1 are used as guide bars, to simply implement the pressing operation, so that the push rod reset lever 4 does not require setting of guide bars, which simplifies the structure; and during sliding, the extension pieces 1 can be restored to a pre-dilation state, as shown in FIG. 14, when the rod is pressed to the bottom of the U-shaped groove 21, the cannula 412 of the pressure lever 41 sleeves the sidewall of the U-shaped groove 21 externally, to implement a rigid pressing operation.

It can be imaged that the handheld portion 42 may also be of another structure to restrict the extension pieces 1 to move along a push rod direction, for example, corresponding threaded holes are disposed on them, to achieve fixed connection, or pin plug or other manners. Certainly, the snapping manner of the boss 13 and the side hole 22 is much simpler.

To prevent the reset rod 5 from rotating or being damaged during pressing, one end on the pressure lever 41 provided with the cannula 412 can be provided with a concave arc surface that fits with a surface of the reset rod 5, as shown in FIGS. 13 and 14.

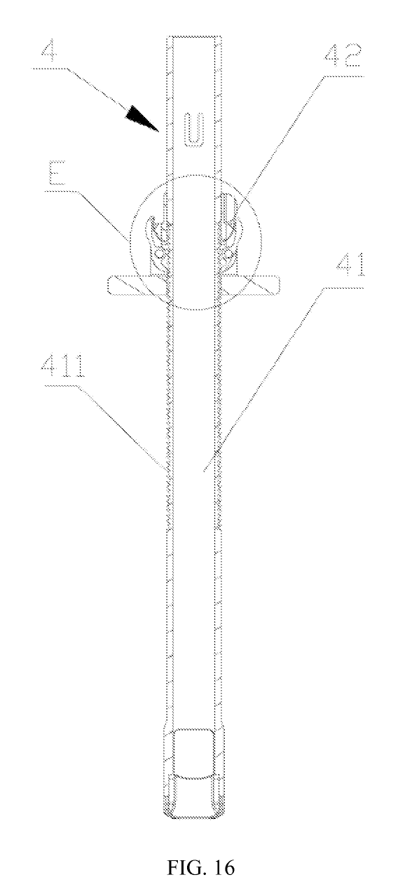

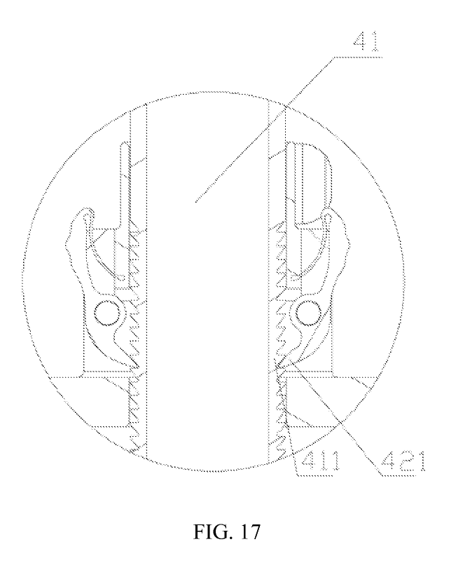

In addition, an outer wall of the pressure lever 41 can be provided with pawls 411, and the handheld portion 42 is provided with latches 421 that can be snapped into gaps between adjacent pawls 411, as shown in FIGS. 16-17, FIG. 16 is an axial sectional view of the push rod reset lever in FIG. 12; and FIG. 17 is a schematic partially enlarged view of Site E in FIG. 16.

The pawls 411 are backed towards a direction of the reset rod 5, the latches 421 of the handheld portion 42 do not hinder pressing of the pressure lever 41, but cooperation of the latches 421 and the pawls 411 makes that the pressure lever 41 cannot move upward, and can only be pressed down, the structure helps to ensure the pressing effect, and when the pressure lever 41 presses the reset rod 5 completely to the bottom of the U-shaped groove 21, the pressure lever 41 and the pedicle screw 2 cannot move relatively, to form rigid constraint cooperation, thereby ensuring the subsequent resetting operation (stretch/pressurization); after the surgery, the push rod reset lever 4 and the extension pieces 1 can be destructively removed through specialized tools.

The extension piece at a screw end of a pedicle screw, the dilator and the push rod reset lever according to the present invention are described in detail above. The principle and implementation manners of the present invention are elaborated herein with specific examples, and description of the embodiments is only used for helping to understand the method of the present invention and its core ideas. It should be noted that, persons of ordinary skill in the art may make improvements and modifications to the present invention without departing from the principle of the present invention, which fall within the protection scope of the claims of the present invention.

1. An extension piece at a screw end of a pedicle screw, two extension pieces being oppositely disposed at the tail of the pedicle screw, wherein the extension pieces are flexible extension pieces.

2. The extension piece according to claim 1, wherein one end of the extension pieces is provided with a snap hole for snap-in of a transverse boss at the screw end of the pedicle screw; the end of the extension pieces is further provided with a flexible snap strip opposite to the snap hole, and the snap strip forms a snap ring for snap-in of an axial boss at the screw end of the pedicle screw with two sides of the extension pieces.

3. The extension piece according to claim 2, wherein two sides of one end of the extension pieces are both provided with a bending sidewall, the end being one end of the extension pieces provided with the snap hole; the snap strip forms the snap ring with the two bending sidewalls; and contact surfaces of the snap strip and the axial boss at the screw end of the pedicle screw are matched with each other.

4. The extension piece according to claim 1, wherein an end portion of the extension pieces away from the pedicle screw is provided with a boss extending outwardly.

5. A dilator for dilating the extension piece according claim 1, wherein the dilator comprises two hinged rods; each of the rods comprises a dilation head and a dilation leg connected with each other, and their connection position is a hinge position; the two dilation legs can extend between the two extension pieces, and the two dilation heads can open and close by external force to drive ends of the dilation legs to be near or away; and portions of the two extension pieces inside the skin can be dilated and separated when the ends of the dilation legs are away.

6. The dilator according to claim 5, wherein a spring is disposed between the two dilation heads.

7. The dilator according to claim 6, wherein the two dilation heads are both connected with a dilation handle.

8. The dilator according to claim 7, further comprising a housing covering the dilation heads, the spring and the hinge position, two sides of the housing being provided with a snap cover for insertion of the pieces.

9. The dilator according to claim 5, wherein the ends of the dilation legs each have a clamp for insertion of the extension pieces, and the clamp has an opening.

10. The dilator according to claim 5, wherein the two dilation legs are crossed.

11. A push rod reset lever, cooperating with the extension piece according to claim 1 to press a reset rod, wherein the push rod reset lever comprises a pressure lever and a handheld portion that can restrict the two extension pieces to move along a push rod direction, the pressure lever can be inserted into the handheld portion and slides axially relative to the handheld portion; and two sides of one end of the pressure lever are both provided with a cannula, respectively for insertion of the two extension pieces and screw groove sidewalls of the pedicle screw.

12. The push rod reset lever according to claim 11, wherein an outer wall of the pressure lever is provided with pawls, and the handheld portion is provided with latches that can be snapped into gaps between adjacent pawls.

13. The push rod reset lever according to claim 11, wherein two sides of the handheld portion are both provided with a side hole, an end portion of the extension pieces away from the pedicle screw is provided with a boss extending outwardly, and the side hole of the handheld portion is for snap-in of the boss.

14. The push rod reset lever according to claim 11, wherein one end of the pressure lever provided with the cannula has a concave arc surface that fits with a surface of the reset rod.
